SEQUENCE LISTING (11 sequences)

SEQ ID NO: 1 - DNA, 3480 bp, Homo sapiens
ctccctctttccctcacagccgacgaggcaacaattaggctttggggataaaacgaggtgcggagagcgggctggggcatttctccccgagatggcgggtctgacggcggcggccccgcggcccggagtcctcctgctcctgctgtccatcctccacccctctcggcctggaggggtccctggggccattcctggtggagttcctggaggagtcttttatccaggggctggtctcggagcccttggaggaggagcgctggggcctggaggcaaacctcttaagccagttcccggagggcttgcgggtgctggccttggggcagggctcggcgccttccccgcagttacctttccgggggctctggtgcctggtggagtggctgacgctgctgcagcctataaagctgctaaggctggcgctgggcttggtggtgtcccaggagttggtggcttaggagtgtctgcaggtgcggtggttcctcagcctggagccggagtgaagcctgggaaagtgccgggtgtggggctgccaggtgtatacccaggtggcgtgctcccaggagctcggttccccggtgtgggggtgctccctggagttcccactggagcaggagttaagcccaaggctccaggtgtaggtggagcttttgctggaatcccaggagttggaccctttgggggaccgcaacctggagtcccactggggtatcccatcaaggcccccaagctgcctggtggctatggactgccctacaccacagggaaactgccctatggctatgggcccggaggagtggctggtgcagcgggcaaggctggttacccaacagggacaggggttggcccccaggcagcagcagcagcggcagctaaagcagcagcaaagttcggtgctggagcagccggagtcctccctggtgttggaggggctggtgttcctggcgtgcctggggcaattcctggaattggaggcatcgcaggcgttgggactccagctgcagctgcagctgcagcagcagccgctaaggcagccaagtatggagctgctgcaggcttagtgcctggtgggccaggctttggcccgggagtagttggtgtcccaggagctggcgttccaggtgttggtgtcccaggagctgggattccagttgtcccaggtgctgggatcccaggtgctgcggttccaggggttgtgtcaccagaagcagctgctaaggcagctgcaaaggcagccaaatacggggccaggcccggagtcggagttggaggcattcctacttacggggttggagctgggggctttcccggctttggtgtcggagtcggaggtatccctggagtcgcaggtgtccctggtgtcggaggtgttcccggagtcggaggtgtcccgggagttggcatttcccccgaagctcaggcagcagctgccgccaaggctgccaagtacggagtggggaccccagcagctgcagctgctaaagcagccgccaaagccgcccagtttgggttagttcctggtgtcggcgtggctcctggagttggcgtggctcctggtgtcggtgtggctcctggagttggcttggctcctggagttggcgtggctcctggagttggtgtggctcctggcgttggcgtggctcccggcattggccctggtggagttgcagctgcagcaaaatccgctgccaaggtggctgccaaagcccagctccgagctgcagctgggcttggtgctggcatccctggacttggagttggtgtcggcgtccctggacttggagttggtgctggtgttcctggacttggagttggtgctggtgttcctggcttcggggcagtacctggagccctggctgccgctaaagcagccaaatatggagcagcagtgcctggggtccttggagggctcggggctctcggtggagtaggcatcccaggcggtgtggtgggagccggacccgccgccgccgctgccgcagccaaagctgctgccaaagccgcccagtttggcctagtgggagccgctgggctcggaggactcggagtcggagggcttggagttccaggtgttgggggccttggaggtatacctccagctgcagccgctaaagcagctaaatacggtgctgctggccttggaggtgtcctagggggtgccgggcagttcccacttggaggagtggcagcaagacctggcttcggattgtctcccattttcccaggtggggcctgcctggggaaagcttgtggccggaagagaaaatgagcttcctaggacccctgactcacgacctcatcaacgttggtgctactgcttggtggagaatgtaaaccctttgtaaccccatcccatgcccctccgactccccaccccaggagggaacgggcaggccgggcggccttgcagatccacagggcaaggaaacaagaggggagcggccaagtgccccgaccaggaggccccctacttcagaggcaagggccatgtggtcctggccccccaccccatcccttcccacctaggagctccccctccacacagcctccatctccaggggaacttggtgctacacgctggtgctcttatcttcctggggggagggaggagggaagggtggcccctcggggaaccccctacctggggctcctctaaagatggtgcagacacttcctgggcagtcccagctccccctgcccaccaggacccaccgttggctgccatccagttggtacccaagcacctgaagcctcaaagctggattcgctctagcatccctcctctcctgggtccacttggccgtctcctccccaccgatcgctgttccccacatctggggcgcttttgggttggaaaaccaccccacactgggaatagccaccttgcccttgtagaatccatccgcccatccgtccattcatccatcggtccgtccatccatgtccccagttgaccgcccggcaccactagctggctgggtgcacccaccatcaacctggttgacctgtcatggccgcctgtgccctgcctccacccccatcctacactcccccagggcgtgcggggctgtgcagactggggtgccaggcatctcctccccacccggggtgtccccacatgcagtactgtataccccccatccctccctcggtccactgaacttcagagcagttcccattcctgccccgcccatctttttgtgtctcgctgtgatagatcaataaatattttattttttgtcctggatatttggggattatttttgattgttgatattctcttttggttttattgttgtggttcattgaaaaaaaaagataatttttttttctgatccggggagctgtatccccagtagaaaaaacattttaatcactctaatataactctggatgaaacacacctttttttttaataagaaaagagaattaactgcttcagaaatgactaataaatgaaaaacctttaaaggaaaaaaaaaa

SEQ ID NO: 2 - DNA, 1403 bp, Homo sapiens
gcctgtctgcattctactatataaagcagcagagacgttgactagcgcatatttgctaagagcaccatgcgcgcagcagccatctccactccaaagttagacaaaatgccaggaatgttcttctctgctaacccaaaggaattgaaaggaaccactcattcacttctagacgacaaaatgcaaaaaaggaggccaaagacttttggaatggatatgaaagcatacctgagatctatgatcccacatctggaatctggaatgaaatcttccaagtccaaggatgtactttctgctgctgaagtaatgcaatggtctcaatctctggaaaaacttcttgccaaccaaactggtcaaaatgtctttggaagtttcctaaagtctgaattcagtgaggagaatattgagttctggctggcttgtgaagactataagaaaacagagtctgatcttttgccctgtaaagcagaagagatatataaagcatttgtgcattcagatgctgctaaacaaatcaatattgacttccgcactcgagaatctacagccaagaagattaaagcaccaacccccacgtgttttgatgaagcacaaaaagtcatatatactcttatggaaaaggactcttatcccaggttcctcaaatcagatatttacttaaatcttctaaatgacctgcaggctaatagcctaaagtgactggtccctggctgaagggaattaacagatagtatcaagcgcagaaggaatgtgccagtatggctccctgggtgaacagcttggccttttttgggtgtcttgacaggccaagaagaacaaatgactcagaatggattaacatgaaagttatccaggcgcagagttgaagaagcataagcaagacaaaaacagagagaccgcagaaggaggaagatactgtggtactgtcataaaaaacagtggagctctgtattagaaagcccctcagaactgggaaggccaggtaactctagttacacagaaactgtgactaaagtctatgaaactgattacaacagactgtaagaatcaaagtcaactgacatctatgctacatattattatatagtttgtactgagctattgaagtcccattaacttaaagtatatgttttcaaattgccattgctactattgcttgtcggtgttattttattttattgtttttgactttggaagagatgaactgtgtatttaacttaagctattgctcttaaaaccagggagtcagaatatatttgtaagttaaatcattggtgctaataataaatgtggattttgtattaaaatatatagaagcaatttctgtttacatgtccttgctacttttaaaaacttgcatttattcctcagattttaaaaataaataaataattcatttaagattc

SEQ ID NO: 3 - DNA, 2746 bp, Homo sapiens
ggctccgacttggactccctgctccgctgctgccgcttcggccccgcacgcagccagccgccagccgcccgcccggcccagctcccgccgcggccccttgccgcggtccctctcctggtcccctcccggttggtccgggggtgcgcagggggcagggcgggcgcccaggggaagctcgagggacgcgcgcgcgaaggctcctttgtggacttcacggccgccaacatctgggcgcagcgcgggccaccgctggccgtctcgccgccgcgtcgccttggggacccgagggggctcagccccaaggacggagacttcgattcgggaccagccccccgggatgcggtagcggccgctgtgcggaggccgcgaagcagctgcagccgccgccgcgcagatccacgctggctccgtgcgccatggtcacccacagcaagtttcccgccgccgggatgagccgccccctggacaccagcctgcgcctcaagaccttcagctccaagagcgagtaccagctggtggtgaacgcagtgcgcaagctgcaggagagcggcttctactggagcgcagtgaccggcggcgaggcgaacctgctgctcagtgccgagcccgccggcacctttctgatccgcgacagctcggaccagcgccacttcttcacgctcagcgtcaagacccagtctgggaccaagaacctgcgcatccagtgtgaggggggcagcttctctctgcagagcgatccccggagcacgcagcccgtgccccgcttcgactgcgtgctcaagctggtgcaccactacatgccgccccctggagccccctccttcccctcgccacctactgaaccctcctccgaggtgcccgagcagccgtctgcccagccactccctgggagtccccccagaagagcctattacatctactccgggggcgagaagatccccctggtgttgagccggcccctctcctccaacgtggccactcttcagcatctctgtcggaagaccgtcaacggccacctggactcctatgagaaagtcacccagctgccggggcccattcgggagttcctggaccagtacgatgccccgctttaaggggtaaagggcgcaaagggcatgggtcgggagaggggacgcaggcccctctcctccgtggcacatggcacaagcacaagaagccaaccaggagagagtcctgtagctctggggggaaagagggcggacaggcccctccctctgccctctccctgcagaatgtggcaggcggacctggaatgtgttggagggaagggggagtaccacctgagtctccagcttctccggaggagccagctgtcctggtgggacgatagcaaccacaagtggattctccttcaattcctcagcttcccctctgcctccaaacaggggacacttcgggaatgctgaactaatgagaactgccagggaatcttcaaactttccaacggaacttgtttgctctttgatttggtttaaacctgagctggttgtggagcctgggaaaggtggaagagagagaggtcctgagggccccagggctgcgggctggcgaaggaaatggtcacaccccccgcccaccccaggcgaggatcctggtgacatgctcctctccctggctccggggagaagggcttggggtgacctgaagggaaccatcctggtaccccacatcctctcctccgggacagtcaccgaaaacacaggttccaaagtctacctggtgcctgagagcccagggcccttcctccgttttaagggggaagcaacatttggaggggatggatgggctggtcagctggtctccttttcctactcatactataccttcctgtacctgggtggatggagcgggaggatggaggagacgggacatctttcacctcaggctcctggtagagaagacaggggattctactctgtgcctcctgactatgtctggctaagagattcgccttaaatgctccctgtcccatggagagggacccagcataggaaagccacatactcagcctggatgggtggagaggctgagggactcactggagggcaccaagccagcccacagccagggaagtggggagggggggcggaaacccatgcctcccagctgagcactgggaatgtcagcccagtaagtattggccagtcaggcgcctcgtggtcagagcagagccaccaggtcccactgccccgagccctgcacagccctccctcctgcctgggtgggggaggctggaggtcattggagaggctggactgctgccaccccgggtgctcccgctctgccatagcactgatcagtgacaatttacaggaatgtagcagcgatggaattacctggaacagttttttgtttttgtttttgtttttgtttttgtgggggggggcaactaaacaaacacaaagtattctgtgtcaggtattgggctggacagggcagttgtgtgttggggtggtttttttctctatttttttgtttgtttcttgttttttaataatgtttacaatctgcctcaatcactctgtcttttataaagattccacctccagtcctctctcctcccccctactcaggcccttgaggctattaggagatgcttgaagaactcaacaaaatcccaatccaagtcaaactttgcacatatttatatttatattcagaaaagaaacatttcagtaatttataataaagagcactattttttaatgaaaaaaaaaaaaaaaaa

SEQ ID NO: 4 - DNA, 4507 bp, Homo sapiens
gaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataatgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatatcttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactatttaaaaattgacagatcttttctgaagaaaaaaaaaaaaaaa

SEQ ID NO: 5 - DNA, 3338 bp, Homo sapiens
gacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaaaaaaaaaaaaaaaaa

SEQ ID NO: 6 - DNA, 2400 bp, Homo sapiens
tccgctccgttcggccggttctcccgggaagctattaatagcattacgtcagcctgggactggcaacacggagtaaacgaccgcgccgccagcctgagggctataaaaggggtgatgcaacgctctccaagccacagtcgcacgcagccaggcgcgcactgcacagctctcttctctcgccgccgcccgagcgcacccttcagcccgcgcgccggccgtgagtcctcggtgctcgcccgccggccagacaaacagcccgcccgaccccgtcccgaccctggccgccccgagcggagcctggagcaaaatgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaactcccaaggcccttttgggtccagaagacctgcatatgggctgttgactcatgcaaatgaggtatctgaactgcagcttcagtattagcagagccacaggccgcctctgtggcatcaccagggtttctctgaagaagagggtctgcattttcctaaacccagtgctgctctcccatctcccatcttcctctcgcagcttgatgagccccggtgtgtcccaggtacacccctgcatccaggcagcagcccaggccaccccctcctcactggcccttggctcctttcttgatgcctctgttgcttgtcccccaggagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagctaagcagtcgtggtatgggggcgactggggagtcctcattgaatcctcattttatacccaaaaccctgaagccattggagagctgtcttcctgtgtacctctagaatcccagcagcagagaaccatcaaggcgggagggcctgcagtgattcagcaggcccttcccattctgccccagagtgggtcttggaccagggcaagtgcatctttgcctcaactccaggatttaggccttaacacactggccattcttatgttccagatggcccccagctggtgtcctgcccgcctttcatctggattctacaaaaaaccaggatgcccaccgttaggattcaggcagcagtgtctgtacctcgggtgggagggatggggccatctccttcaccgtggctaccattgtcactcgtaggggatgtggagtgagaacagcatttagtgaagttgtgcaacggccagggttgtgctttctagcaaatatgctgttatgtccagaaattgtgtgtgcaagaaaactaggcaatgtactcttccgatgtttgtgtcacacaacactgatgtgacttttatatgctttttctcagatctggtttctaagagttttggggggcggggctgtcaccacgtgcagtatctcaagatattcaggtggccagaagagcttgtcagcaagaggaggacagaattctcccagcgttaacacaaaatccatgggcagtatgatggcaggtcctctgttgcaaactcagttccaaagtcacaggaagaaagcagaaagttcaacttccaaagggttaggactctccactcaatgtcttaggtcaggagttgtgtctaggctggaagagccaaagaatattccattttcctttccttgtggttgaaaaccacagtcagtggagagatgtttggaaaccacagtcagtggagcctgggtggtacccaggctttagcattattggatgtcaatagcattgtttttgtcatgtagctgttttaagaaatctggcccagggtgtttgcagctgtgagaagtcactcacactggccacaaggacgctggctactgtctattaaaattctgatgtttctgtgaaattctcagagtgtttaattgtactcaatggtatcattacaattttctgtaagagaaaatattacttatttatcctagtattcctaacctgtcagaataataaatattggaaccaagacatggtaaacaaaaaaaaaaaaaa

SEQ ID NO: 7 - DNA, 2358 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 8 - DNA, 5182 bp, Homo sapiens
cgcctgtccccctcccgaggcccgggctcgcgacggcagagggctccgtcggcccaaaccgagctgggcgcccgcggtccgggtgcagcctccactccgccccccagtcaccgcctcccccggcccctcgacgtggcgcccttccctccgcttctctgtgctccccgcgcccctcttggcgtctggccccggcccccgctctttctcccgcaaccttcccttcgctccctcccgtcccccccagctcctagcctccgactccctccccccctcacgcccgccctctcgccttcgccgaaccaaagtggattaattacacgctttctgtttctctccgtgctgttctctcccgctgtgcgcctgcccgcctctcgctgtcctctctccccctcgccctctcttcggcccccccctttcacgttcactctgtctctcccactatctctgcccccctctatccttgatacaacagctgacctcatttcccgataccttttcccccccgaaaagtacaacatctggcccgccccagcccgaagacagcccgtcctccctggacaatcagacgaattctccccccccccccaaaaaaaagccatccccccgctctgccccgtcgcacattcggcccccgcgactcggccagagcggcgctggcagaggagtgtccggcaggagggccaacgcccgctgttcggtttgcgacacgcagcagggaggtgggcggcagcgtcgccggcttccagacaccaatgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtgagcaaaactgccgcaagtctgcagcccggcgccaccatcctgcagcctcctcctgaccacggacgtttccatcaggttccatcccgaaaatctctcggttccacgtccccctggggcttctcctgacccagtccccgtgccccgcctccccgaaacaggctactctcctcggccccctccatcgggctgaggaagcacagcagcatcttcaaacatgtacaaaatcgattggctttaaacacccttcacataccctccccccaaattatccccaattatccccacacataaaaaatcaaaacattaaactaacccccttcccccccccccacaacaaccctcttaaaactaattggctttttagaaacaccccacaaaagctcagaaattggctttaaaaaaaacaaccaccaaaaaaaatcaattggctaaaaaaaaaaagtattaaaaacgaattggctgagaaacaattggcaaaataaaggaatttggcactccccacccccctctttctcttctcccttggactttgagtcaaattggcctggacttgagtccctgaaccagcaaagagaaaagaaggaccccagaaatcacaggtgggcacgtcgctgctaccgccatctcccttctcacgggaattttcagggtaaactggccatccgaaaatagcaacaacccagactggctcctcactcccttttccatcactaaaaatcacagagcagtcagagggacccagtaagaccaaaggaggggaggacagagcatgaaaaccaaaatccatgcaaatgaaatgtaattggcacgaccctcacccccaaatcttacatctcaattcccatcctaaaaagcactcatactttatgcatccccgcagctacacacacacaacacacagcacacgcatgaacacagcacacacacgagcacagcacacacacaaacgcacagcacacacagcacacagatgagcacacagcacacacacaaacgcacagcacacacacgcacacacatgcacacacagcacacaaacgcacggcacacacacgcacacacatgcacacacagcacacacacaaacgcacagcacacacaaacgcacagcacacacgcacacacagcacacacacgagcacacagcacacaaacgcacagcacacgcacacacatgcacacacagcacacacactagcacacagcacacacacaaagacacagcacacacatgcacacacagcacacacacgcgaacacagcacacacgaacacagcacacacagcacacacacaaacacagcacacacatgcacacagcacacgcacacacagcacacacatgaacacagcacacagcacacacatgcacacacagcacacacgcatgcacagcacacatgaacacagcacacacacaaacacacagcacacacatgcacacacagcacacacactcatgcgcagcacatacatgaacacagctcacagcacacaaacacgcagcacacacgttgcacacgcaagcacccacctgcacacacacatgcgcacacacacgcacacccccacaaaattggatgaaaacaataagcatatctaagcaactacgatatctgtatggatcaggccaaagtcccgctaagattctccaatgttttcatggtctgagccccgctcctgttcccatctccactgcccctcggccctgtctgtgccctgcctctcagaggagggggctcagatggtgcggcctgagtgtgcggccggcggcatttgggatacacccgtagggtgggcggggtgtgtcccaggcctaattccatctttccaccatgacagagatgcccttgtgaggctggcctccttggcgcctgtccccacggcccccgcagcgtgagccacgatgctccccataccccacccattcccgatacaccttacttactgtgtgttggcccagccagagtgaggaaggagtttggccacattggagatggcggtagctgagcagacatgcccccacgagtagcctgactccctggtgtgctcctggaaggaagatcttggggacccccccaccggagcacacctagggatcatctttgcccgtctcctggggaccccccaagaaatgtggagtcctcgggggccgtgcactgatgcggggagtgtgggaagtctggcggttggaggggtgggtggggggcagtgggggctgggcggggggagttctggggtaggaagtggtcccgggagattttggatggaaaagtcaggaggattgacagcagacttgcagaattacatagagaaattaggaacccccaaatttcatgtcaattgatctattccccctctttgtttcttggggcatttttccttttttttttttttttgttttttttttacccctccttagctttatgcgctcagaaaccaaattaaaccccccccccatgtaacaggggggcagtgacaaaagcaagaacgcacgaagccagcctggagaccaccacgtcctgccccccgccatttatcgccctgattggattttgtttttcatctgtccctgttgcttgggttgagttgagggtggagcctcctggggggcactggccactgagcccccttggagaagtcagaggggagtggagaaggccactgtccggcctggcttctggggacagtggctggtccccagaagtcctgagggcggaggggggggttgggcagggtctcctcaggtgtcaggagggtgctcggaggccacaggagggggctcctggctggcctgaggctggccggaggggaaggggctagcaggtgtgtaaacagagggttccatcaggctggggcagggtggccgccttccgcacacttgaggaaccctcccctctccctcggtgacatcttgcccgcccctcagcaccctgccttgtctccaggaggtccgaagctctgtgggacctcttgggggcaaggtggggtgaggccggggagtagggaggtcaggcgggtctgagcccacagagcaggagagctgccaggtctgcccatcgaccaggttgcttgggccccggagcccacgggtctggtgatgccatagcagccaccaccgcggcgcctagggctgcggcagggactcggcctctgggaggtttacctcgcccccacttgtgcccccagctcagcccccctgcacgcagcccgactagcagtctagaggcctgaggcttctgggtcctggtgacggggctggcatgaccccgggggtcgtccatgccagtccgcctcagtcgcagagggtccctcggcaagcgccctgtgagtgggccattcggaacattggacagaagcccaaagagccaaattgtcacaattgtggaacccacattggcctgagatccaaaacgcttcgaggcaccccaaattacctgcccattcgtcaggacacccacccacccagtgttatattctgcctcgccggagtgggtgttcccgggggcacttgccgaccagccccttgcgtccccaggtttgcagctctcccctgggccactaaccatcctggcccgggctgcctgtctgacctccgtgcctagtcgtggctctccatcttgtctcctccccgtgtccccaatgtcttcagtggggggccccctcttgggtcccctcctctgccatcacctgaagacccccacgccaaacactgaatgtcacctgtgcctgccgcctcggtccaccttgcggcccgtgtttgactcaactcaactcctttaacgctaatatttccggcaaaatcccatgcttgggttttgtctttaaccttgtaacgcttgcaatcccaataaagcattaaaagtcatgaaaaaaaaaaaaaaaaaa

SEQ ID NO: 9 - DNA, 4305 bp, Homo sapiens
agatgccgcctggcaccaagcgcagccgccgctgccgcactttccacttgtattgatcacctctcagccccgcgcagccggctcgcccgagcggaccgcggccagcgcgccagcccttggcagccccggagcagtcgggctccgggaggaaactccttgggagcgccctgtccggggtgccctctgcgctctgcagtgtctttctttctgcctgggaggaggaggaggaggaggaagaggaggaggaggaggaggaggaggaggaagaggaggaggaggaggaggacgtctggtcccggctgggaggtggagcagcggcagcagcagcagccgccgccgccgccgccgctgccgccgccgccggaaagggagaggcaggagagcccgagacttggaaaccccaaagtgtccgcgaccctgcacggcaggctcccttccagcttcatgggcaaagtgtggaaacagcagatgtaccctcagtacgccacctactattacccccagtatctgcaagccaagcagtctctggtcccagcccaccccatggcccctcccagtcccagcaccaccagcagtaataacaacagtagcagcagtagcaactcaggatgggatcagctcagcaaaacgaacctctatatccgaggactgcctccccacaccaccgaccaggacctggtgaagctctgtcaaccatatgggaaaatagtctccacaaaggcaattttggataagacaacgaacaaatgcaaaggttatggttttgtcgactttgacagccctgcagcagctcaaaaagctgtgtctgccctgaaggccagtggggttcaagctcaaatggcaaagcaacaggaacaagatcctaccaacctctacatttctaatttgccactctccatggatgagcaagaactagaaaatatgctcaaaccatttggacaagttatttctacaaggatactacgtgattccagtggtacaagtcgtggtgttggctttgctaggatggaatcaacagaaaaatgtgaagctgttattggtcattttaatggaaaatttattaagacaccaccaggagtttctgcccccacagaacctttattgtgtaagtttgctgatggaggacagaaaaagagacagaacccaaacaaatacatccctaatggaagaccatggcatagagaaggagaggtgagacttgctggaatgacacttacttacgacccaactacagctgctatacagaacggattttatccttcaccatacagtattgctacaaaccgaatgatcactcaaacttctattacaccctatattgcatctcctgtatctgcctaccaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaacctaataagtaactgtgagatgtacagaaaggtgttcttacatgaagaagggtgtgaaggctgaacaatcatggatttttctgatcaattgtgctttaggaaattattgacagttttgcacaggttcttgaaaacgttatttataatgaaatcaactaaaactatttttgctataagttctataaggtgcataaaacccttaaattcatctagtagctgttcccccgaacaggtttattttagtaaaaaaaaaaaaacaaaaaacaaaaacaaaagatttttatcaaatgttatgatgcaaaaaaagaaaaagaaaaaaaaaaagaaaagaaaacttcaattttctgggtatgcacaaagaccatgaagacttatccaagtgcatgaccggatttttgtggttttgttcattttgtgtttaatttgtgttttttttttccagctgtatgaaatgggctttctgaagtttaaatagtccgacttcacccatggtgttctgtgcttgcagtgcgagtgttgctgtaattcagtgttgccgtcagtgtctcttttcttagctttctgtctttctttcaacgtagtgtgaagtgtcttatccttttctatgaattccaatttgccttaactcttttgatgctgtagctgtttcagtaaaagttagttcaaactaatgatgtagaatgctttgaccaaatgagctggtctattatgccttgtaaaacagcagcatagggcttttaaaaggtagtcaataaaagttgctgaaattttggcttttttaaatatgtagtaggtgtttttaatgatttttcacataatgtgtaaggtagtgaaatgcaagaagggaaaaatgttttgtgtgaaacacattttctgactggggaacttttattagggtaaattgtttgtaaggctgtacgccaacagtttcctctgatagtttgactgatttaggatatctgctgtatgatgcaatgtaaagtcttttttgccttttttcaggaaaaaaaaaaagctaacttgatgtactagatttagtgtaggtagtgttggggttggggatgggggtgggggaggggagtcactgaatgttttgtccttcctttatactaatgatagtgctttagaatgagaattatgcctgaaatctggcaaaccgaaaaatgttgctattgcaacaaagtggcaaaagctaaaagtaaggatttatcttcaaacataagctgagataacgaatagaagcaaaacgattggctactagctctctctctctctctctattaggtaaatttgaaaaataaaaatgacttggcacttttaaaggtaacttcaccaaagaccgaagagccagtaaccagtagctccaacttgtctcagcatcacatcttctgtgctctttatttttgccggaccagtttgcggttaggagaatgtgccttttttgtacctttgcatttaggttttataattttaattgatgtatggacacacacaaacaaaaaagcatgaaggaagatttggatccaagcagtgccacactttacatcatcactacaagtgttcaagtgtaaagaaaaccaattttgaaactatgaaattcctgattcataaatacacagttatttctactttagtacatataagataattcactgttattaaagctcttttattaaggcaattgcatatgttttaaaagcaatggtaaattaagttgtcttccaaaactgtgtacttgtctggtcagctgtgtatgatcagttatctacctcagagtctattttcttttgtgctgggacaggttgctggccctccctgtttccacagaccaaatcctcctagctcaggagctagggctaagcagttatttctttcaagtattttttagttcttaaattttatgcttgtatttgatgatagatgtcagtgacatttcatagtttcaaaagtccttgctgctctgagaagtgtagattctagtgaaaattacatagtcataagagaaatgtgtttttgtttttgtttttgtttcatttttttaaagttgtggtattattggttctatgctccctggaatattactgctttgtgaaagtccagactgaacgcagcaccctctgtgtacctagtacagttataaacctgggtctctcactacttgatatttttgcattagttaagacagaaatttgatagctcggttagaggggaggggaaatctgctgctagaaatgtctgaactaagtgccatactcgtctgggtaagatttgggaaacataacctctgtacataaaaaaaaaaaaatcagttaaacatcacatagtagacagccattaaattataaaaaaattaatttatgaagaaagaccttttgtacagattgaaaaaaaaagattttcatagagatatctatatgatcaagagagttaattttttatttttgttttactagtgccacagacttgccagtggtaacttatttgtccggttcaagataactctgtagttttctttcctaggacttgttgttaaacgccaaaagacatttttgaactgtacatttgatcagattgttagcttttctgttttatttcttttgagaacctttgaataaaaaacatctgaaattttaaaaaaaaaaaaaaaaaaaa

SEQ ID NO: 10 - DNA, 2596 bp, Homo sapiens
caccaagcgcagccgccgctgccgcactttccacttgtattgatcacctctcagccccgcgcagccggctcgcccgagcggaccgcggccagcgcgccagcccttggcagccccggagcagtcgggctccgggaggaaactccttgggagcgccctgtccggggtgccctctgcgctctgcagtgtctttctttctgcctgggaggaggaggaggaggaggaagaggaggaggaggaggaggaggaggaggaagaggaggaggaggaggaggacgtctggtcccggctgggaggtggagcagcggcagcagcagcagccgccgccgccgccgccgctgccgccgccgccggaaagggagaggcaggagagcccgagacttggaaaccccaaagtgtccgcgaccctgcacggcaggctcccttccagcttcatgggcaaagtgtggaaacagcagatgtaccctcagtacgccacctactattacccccagtatctgcaagccaagtttggaaggcattcgggaataccaagtgaaaaggaagagtgaagaacagaaaaatgttagtgggatggcaaacactgtgaacattgctgtttctagtgggccagaaaaatcagtctctggtcccagcccaccccatggcccctcccagtcccagcaccaccagcagtaataacaacagtagcagcagtagcaactcaggatgggatcagctcagcaaaacgaacctctatatccgaggactgcctccccacaccaccgaccaggacctggtgaagctctgtcaaccatatgggaaaatagtctccacaaaggcaattttggataagacaacgaacaaatgcaaaggttatggttttgtcgactttgacagccctgcagcagctcaaaaagctgtgtctgccctgaaggccagtggggttcaagctcaaatggcaaagcaacaggaacaagatcctaccaacctctacatttctaatttgccactctccatggatgagcaagaactagaaaatatgctcaaaccatttggacaagttatttctacaaggatactacgtgattccagtggtacaagtcgtggtgttggctttgctaggatggaatcaacagaaaaatgtgaagctgttattggtcattttaatggaaaatttattaagacaccaccaggagtttctgcccccacagaacctttattgtgtaagtttgctgatggaggacagaaaaagagacagaacccaaacaaatacatccctaatggaagaccatggcatagagaaggagaggtgagacttgctggaatgacacttacttacgacccaactacagctgctatacagaacggattttatccttcaccatacagtattgctacaaaccgaatgatcactcaaacttctattacaccctatattgcatctcctgtatctgcctaccaggtggcaaaggaaaccagagaaaacaagtatcggggctctgctatcaaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaacctaataagtaactgtgagatgtacagaaaggtgttcttacatgaagaagggtgtgaaggctgaacaatcatggatttttctgatcaattgtgctttaggaaattattgacagttttgcacaggttcttgaaaacgttatttataatgaaatcaactaaaactatttttgctataagttctataaggtgcataaaacccttaaattcatctagtagctgttcccccgaacaggtttattttagtaaaaaaaaaaaaacaaaaaacaaaaacaaaagatttttatcaaatgttatgatgcaaaaaaagaaaaagaaaaaaaaaaagaaaagaaaacttcaattttctgggtatgcacaaagaccatgaagacttatccaagtgcatgaccggatttttgtggttttgttcattttgtgtttaatttgtgttttttttttccagctgtatgaaatgggctttctgaagtttaaatagtccgacttcacccatggtgttctgtgcttgcagtgcgagtgttgctgtaattcagtgttgccgtcagtgtctcttttcttagctttctgtctttctttcaacgtagtgtgaagtgtcttatccttttctatgaattccaatttgccttaactcttttgatgctgtagctgtttcagtaaaagttagttcaaactaatgatgtagaatgctttgaccaaatgagctggtctattatgccttgtaaaacagcagcatagggcttttaaaaggtagtcaataaaagttgctgaaattttggcttttttaaaaaaaaaaaaaaaaaa

SEQ ID NO: 11 - DNA, 4296 bp, Homo sapiens
agatgccgcctggcaccaagcgcagccgccgctgccgcactttccacttgtattgatcacctctcagccccgcgcagccggctcgcccgagcggaccgcggccagcgcgccagcccttggcagccccggagcagtcgggctccgggaggaaactccttgggagcgccctgtccggggtgccctctgcgctctgcagtgtctttctttctgcctgggaggaggaggaggaggaggaagaggaggaggaggaggaggaggaggaggaagaggaggaggaggaggaggacgtctggtcccggctgggaggtggagcagcggcagcagcagcagccgccgccgccgccgccgctgccgccgccgccggaaagggagaggcaggagagcccgagacttggaaaccccaaagtgtccgcgaccctgcacggcaggctcccttccagcttcatgggcaaagtgtggaaacagcagatgtaccctcagtacgccacctactattacccccagtatctgcaagccaagcagtctctggtcccagcccaccccatggcccctcccagtcccagcaccaccagcagtaataacaacagtagcagcagtagcaactcaggatgggatcagctcagcaaaacgaacctctatatccgaggactgcctccccacaccaccgaccaggacctggtgaagctctgtcaaccatatgggaaaatagtctccacaaaggcaattttggataagacaacgaacaaatgcaaaggttatggttttgtcgactttgacagccctgcagcagctcaaaaagctgtgtctgccctgaaggccagtggggttcaagctcaaatggcaaagcaacaggaacaagatcctaccaacctctacatttctaatttgccactctccatggatgagcaagaactagaaaatatgctcaaaccatttggacaagttatttctacaaggatactacgtgattccagtggtacaagtcgtggtgttggctttgctaggatggaatcaacagaaaaatgtgaagctgttattggtcattttaatggaaaatttattaagacaccaccaggagtttctgcccccacagaacctttattgtgtaagtttgctgatggaggacagaaaaagagacagaacccaaacaaatacatccctaatggaagaccatggcatagagaaggagaggctggaatgacacttacttacgacccaactacagctgctatacagaacggattttatccttcaccatacagtattgctacaaaccgaatgatcactcaaacttctattacaccctatattgcatctcctgtatctgcctaccaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaacctaataagtaactgtgagatgtacagaaaggtgttcttacatgaagaagggtgtgaaggctgaacaatcatggatttttctgatcaattgtgctttaggaaattattgacagttttgcacaggttcttgaaaacgttatttataatgaaatcaactaaaactatttttgctataagttctataaggtgcataaaacccttaaattcatctagtagctgttcccccgaacaggtttattttagtaaaaaaaaaaaaacaaaaaacaaaaacaaaagatttttatcaaatgttatgatgcaaaaaaagaaaaagaaaaaaaaaaagaaaagaaaacttcaattttctgggtatgcacaaagaccatgaagacttatccaagtgcatgaccggatttttgtggttttgttcattttgtgtttaatttgtgttttttttttccagctgtatgaaatgggctttctgaagtttaaatagtccgacttcacccatggtgttctgtgcttgcagtgcgagtgttgctgtaattcagtgttgccgtcagtgtctcttttcttagctttctgtctttctttcaacgtagtgtgaagtgtcttatccttttctatgaattccaatttgccttaactcttttgatgctgtagctgtttcagtaaaagttagttcaaactaatgatgtagaatgctttgaccaaatgagctggtctattatgccttgtaaaacagcagcatagggcttttaaaaggtagtcaataaaagttgctgaaattttggcttttttaaatatgtagtaggtgtttttaatgatttttcacataatgtgtaaggtagtgaaatgcaagaagggaaaaatgttttgtgtgaaacacattttctgactggggaacttttattagggtaaattgtttgtaaggctgtacgccaacagtttcctctgatagtttgactgatttaggatatctgctgtatgatgcaatgtaaagtcttttttgccttttttcaggaaaaaaaaaaagctaacttgatgtactagatttagtgtaggtagtgttggggttggggatgggggtgggggaggggagtcactgaatgttttgtccttcctttatactaatgatagtgctttagaatgagaattatgcctgaaatctggcaaaccgaaaaatgttgctattgcaacaaagtggcaaaagctaaaagtaaggatttatcttcaaacataagctgagataacgaatagaagcaaaacgattggctactagctctctctctctctctctattaggtaaatttgaaaaataaaaatgacttggcacttttaaaggtaacttcaccaaagaccgaagagccagtaaccagtagctccaacttgtctcagcatcacatcttctgtgctctttatttttgccggaccagtttgcggttaggagaatgtgccttttttgtacctttgcatttaggttttataattttaattgatgtatggacacacacaaacaaaaaagcatgaaggaagatttggatccaagcagtgccacactttacatcatcactacaagtgttcaagtgtaaagaaaaccaattttgaaactatgaaattcctgattcataaatacacagttatttctactttagtacatataagataattcactgttattaaagctcttttattaaggcaattgcatatgttttaaaagcaatggtaaattaagttgtcttccaaaactgtgtacttgtctggtcagctgtgtatgatcagttatctacctcagagtctattttcttttgtgctgggacaggttgctggccctccctgtttccacagaccaaatcctcctagctcaggagctagggctaagcagttatttctttcaagtattttttagttcttaaattttatgcttgtatttgatgatagatgtcagtgacatttcatagtttcaaaagtccttgctgctctgagaagtgtagattctagtgaaaattacatagtcataagagaaatgtgtttttgtttttgtttttgtttcatttttttaaagttgtggtattattggttctatgctccctggaatattactgctttgtgaaagtccagactgaacgcagcaccctctgtgtacctagtacagttataaacctgggtctctcactacttgatatttttgcattagttaagacagaaatttgatagctcggttagaggggaggggaaatctgctgctagaaatgtctgaactaagtgccatactcgtctgggtaagatttgggaaacataacctctgtacataaaaaaaaaaaaatcagttaaacatcacatagtagacagccattaaattataaaaaaattaatttatgaagaaagaccttttgtacagattgaaaaaaaaagattttcatagagatatctatatgatcaagagagttaattttttatttttgttttactagtgccacagacttgccagtggtaacttatttgtccggttcaagataactctgtagttttctttcctaggacttgttgttaaacgccaaaagacatttttgaactgtacatttgatcagattgttagcttttctgttttatttcttttgagaacctttgaataaaaaacatctgaaattttaaaaaaaaaaaaaaaaaaaa

(19) United States
(12) Patent Application Publication
(10) Pub. No.: US 2013/0287701 A1
(43) Pub. Date: Oct. 31, 2013

(54) METHOD OF DETECTING RISK OF CANCER

(71) Applicant: Paul Jenkins, London (GB)

(72) Inventor: Paul Jenkins, London (GB)

(21) Appl. No.: 13/878,096

(22) PCT Filed: Oct. 5, 2011
(86) PCT No.: PCT/GB11/51912

§ 371 Date: May 9, 2013

(30) Foreign Application Priority Data
Oct. 5, 2010 (GB) 1016690.8

(51) Int. Cl. A61K 49/00; C12Q 1/68

(52) U.S. Cl. CPC A61K 49/0058; C12Q 1/6886
USPC 424/9.6; 435/6.12; 435/7.1

(57) ABSTRACT

The invention provides an ex vivo method for detecting the risk of cancer in a patient, comprising the step of: (iii) detecting the expression level of the genes identified herein as ELN, RGS-1, SOCS-3, PTGS-2, JUN, ATF-3, CTGF, IGF-2 and RBMS-1, in a sample of genetic material isolated from a patient, wherein the combined expression level indicates the risk of cancer in the patient from whom the sample was isolated.

### FIELD OF THE INVENTION

This invention relates to methods of detecting the risk of cancer, in particular, colorectal cancer.

### BACKGROUND TO THE INVENTION

Cancer is the second most common cause of death in developed countries, after cardiovascular disease. Colorectal cancer is the second most common cause of cancer death in developed countries, killing 20,000 people a year in the UK.

Screening tests for many types cancer are being introduced by many health providers, but such tests are often not ideal. For example, screening for colorectal cancer usually involves extensive and regular examination of the bowel (colonoscopy) which is uncomfortable, time-consuming, potentially dangerous, has a low pick-up rate and is resource intensive. An alternative screening technique for colorectal cancer is the detection of microscopic amounts of blood in the stool, but this is poorly accepted socially, has a low ‘take-up’ rate and leads to many false-positive results, which consequently require colonoscopy.

The use of molecular diagnostics in cancer aims to use predisposition (or predictive) tests to determine genetic susceptibility. Predictive genetic testing refers to the use of a genetic test in an asymptomatic person to create maps of individual risk and predict future risk of disease. The hope underlying such testing is that early identification of individuals at risk of a specific condition will lead to reduced morbidity and mortality through targeted screening, surveillance, and prevention. Consequently, while conventional diagnostic techniques (including radiography and colonography) indicate whether a tumour is already present, tests that identify genetic aberrations are important to indicate the probability of developing a tumour. This knowledge can help devise the best strategy to prevent the development of a tumour.

The identification of reliable genetic markers for cancer is problematic and, to date, no reliable expression signature has been identified that could be used to predict the risk of colorectal cancer in an individual. There is clearly a need for reliable markers for use in predisposition testing for cancer, in particular colorectal cancer.

### SUMMARY OF THE INVENTION

The present invention is based on the surprising identification of a combination of genetic markers that are useful in predicting the risk of cancer, in particular colorectal cancer.

According to a first aspect of the present invention, an ex vivo method for detecting the risk of cancer in a patient comprises the step of:

(i) detecting the expression level of the genes identified herein as ELN, RGS-1, SOCS-3, PTGS-2, JUN, ATF-3, CTGF, IGF-2 and RBMS-1, in a sample of genetic material isolated from a patient,

wherein the combined expression level indicates the risk of cancer in the patient from whom the sample was isolated.

According to a second aspect, the present invention is directed to the use of a combination of nine isolated genes identified herein as ELN, RGS-1, SOCS-3, PTGS-2, JUN, ATF-3, CTGF, IGF-2 and RBMS-1 in an ex vivo diagnostic assay to test for the risk of cancer in a patient.

According to a third aspect of the invention, a kit for the detection of the risk of cancer in a patient, comprising a combination of reagents that bind to each of the genes identified herein as ELN, RGS-1, SOCS-3, PTGS-2, JUN, ATF-3, CTGF, IGF-2 and RBMS-, and instructions for detecting the risk of cancer.

According to a fourth aspect of the invention, an in vivo method for detecting the risk of cancer in a patient comprises the step of detecting the expression level of genes identified herein as ELN, RGS-1, SOCS-3, PTGS-2, JUN, ATF-3, CTGF, IGF-2 and RBMS-1 in a patient, wherein the expression level indicates the risk of cancer in the patient.

### DESCRIPTION OF THE DRAWINGS

FIG. 1 is a graph showing the relative expression of each of the nine genes normalised with reference genes in both normal normal (NN) and adjacent normal (AN) tissue; and

FIG. 2 is a graph showing the Cancer Risk Index (CRI) calculated from the combined expression level of the nine genes in a cohort of samples in normal normal (NN) and adjacent normal (AN) tissue.

### DESCRIPTION OF THE INVENTION

The present invention is based on the surprising identification of a combination of nine genes that are effective markers for cancer, in particular colorectal cancer. Identification of each of the nine genes, or their expressed products such as mRNA or a polypeptide, in a tissue sample obtained from a patient, preferably a colorectal tissue sample, and comparison of the expression level of the genes with the expression level of the corresponding genes in a control sample indicates the risk of cancer in the patient. This combination of marker genes is therefore useful in predisposition tests for cancer.

The combination of marker genes identified herein is useful in diagnosing the risk of cancer in an individual who has not yet developed the disease, i.e. the marker genes are capable of identifying those individuals who are asymptomatic but who have a genetic predisposition to developing cancer. Such individuals benefit from an early indication of this predisposition as it will allow the regular monitoring of their colorectal tissue, to detect early any potentially cancerous changes.

As used herein, the term “cancer” is to be given its normal meaning in the art, namely a disease characterised by uncontrolled cellular growth and proliferation. The combination of marker genes identified herein is particularly useful in the detection of the risk of colorectal cancer, which is also to be given its usual meaning in the art. For the avoidance of doubt, colorectal cancer refers to cancer that starts in the colon or rectum. The term “colorectal cancer” therefore includes cancers of both the colon and rectum.

As used herein, the terms “patient” and “individual” are used interchangeably and refer to an animal, preferably a mammal, and most preferably a human.

Diagnosis can be made on the basis of the relative expression of the nine genes or gene products in the patient, or patient's sample, compared to control values known levels of expression that are indicative of a patient that is known to be predisposed to cancer. Control values correspond to the relative expression level of each of the nine genes in a corresponding colorectal tissue sample from a non-cancerous individual.

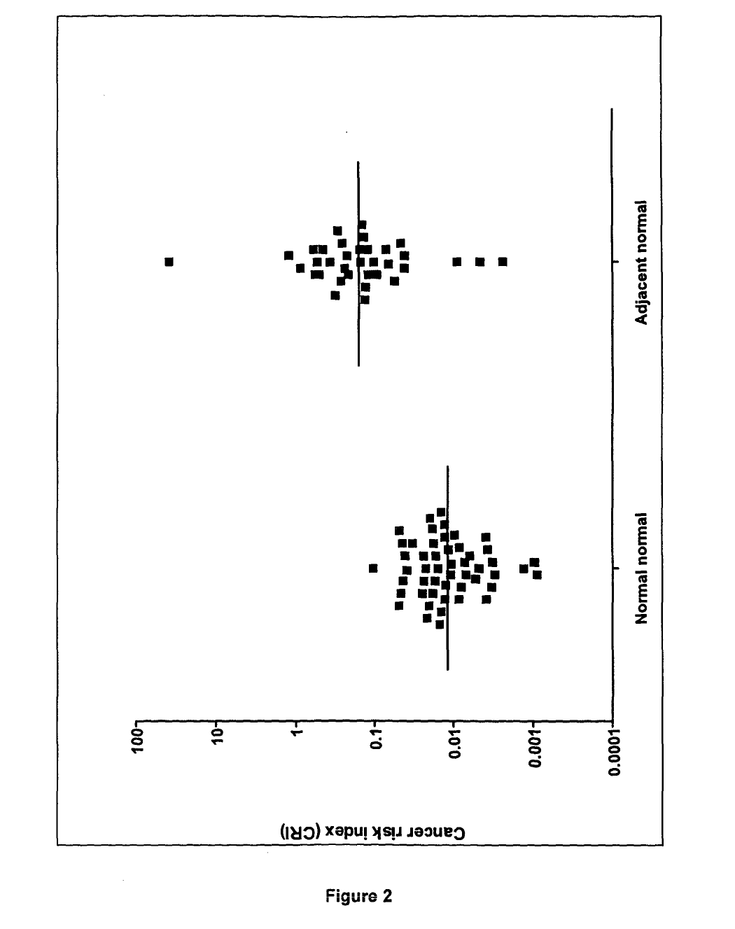

The combined gene expression level may be expressed as single value corresponding to the sum of the expression level of each of the nine genes, which is compared to a single pre-determined control value (calculated from the sum of the expression level of each if the nine genes in a corresponding control sample). In this instance, a combined expression value which is greater than the combined expression value of the control sample indicates a risk of caner in the patient. As such, in order to obtain a positive result it is not necessary for the expression level of all nine genes to be greater than the corresponding gene in the control sample; the result is determined by wither the overall expression value is greater or less then the overall control value. Results calculated in this way are illustrated in FIG. 2.

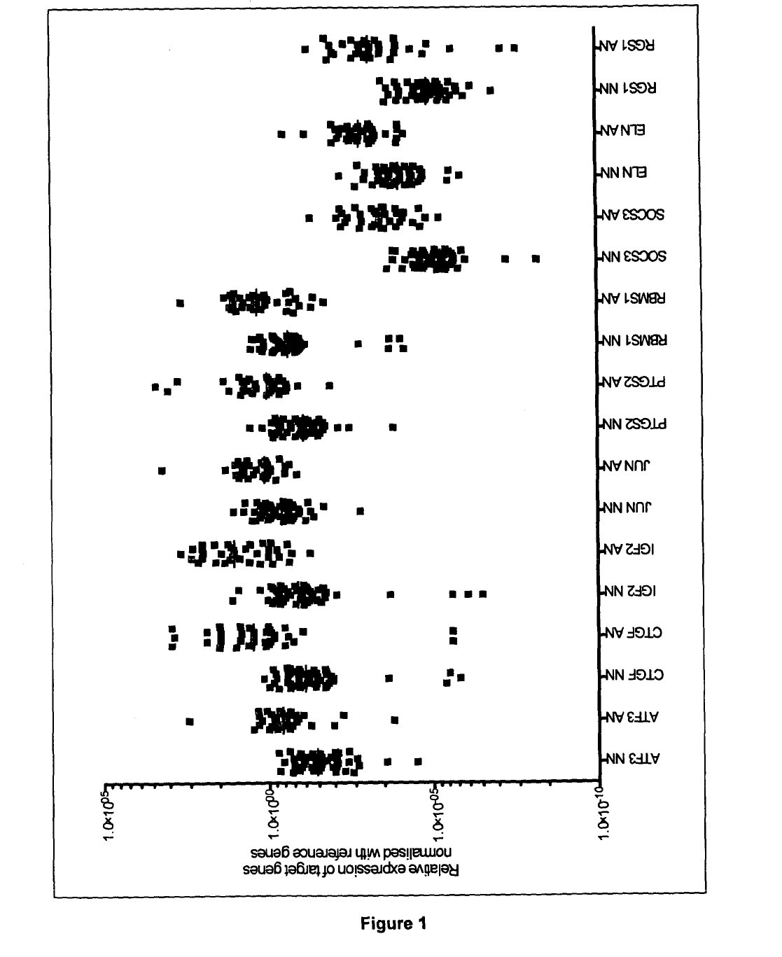

Alternatively, in a preferred embodiment, the method of the invention requires the expression level of each gene to be compared to the expression level of the corresponding gene in a control sample. A positive result for risk of cancer requires expression of each of the nine genes to be up-regulated in the patient or patient sample, compared with the corresponding genes in the control sample. Results calculated in this way are illustrated in FIG. 1.

The marker genes of the present invention are detailed in Table 1, below. The nine marker genes are identified herein as SEQ ID Nos 1-8 and at least one of SEQ ID Nos. 9-11, including complements or fragments thereof that comprise at least 10 consecutive nucleotides, preferably at least 15 consecutive nucleotides, more preferably 30 nucleotides, yet more preferably at least 50 nucleotides and sequences that hybridise to the sequence (or the complement thereof) under stringent hybridising conditions. SEQ ID Nos. 9-11 correspond to three different transcription variants of RBMS-1. The expression level of at least one of these variants is required, in combination with the expression level of each of the sequences identified as SEQ Nos. 1-8, in order to carry out the method of the invention.

Hybridisation will usually be carried out under stringent conditions, known to those in the art, chosen to reduce the possibility of non-complementary hybridisation. Examples of suitable hybridising conditions are disclosed in Nucleic Acid Hybridisation: A Practical Approach (B. D. Hames and S. J. Higgins, editors IRL Press, 1985). An example of stringent hybridisation conditions is overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulphate, and 20 μg/ml denatured, sheared salmon sperm DNA, followed by washing in 0.1×SSC at about 65° C. Homologues of the genes identified herein as SEQ ID Nos. 1-9 are within the scope of the invention. The term “homologue” refers to a sequence that is similar but not identical to one of the identified genes. A homologue performs the same function as the identified gene, i.e. the same biological function. The common name, Genbank accession number and description of each marker sequence is provided in Table 1; a homologue of a marker sequence according to the invention must retain the biological function of the sequence. The biological function of each sequence in Table 1 is known, and is summarised in the “description” column of Table 1. For example, a homologue of SOCS-3 must retain function as a suppressor of cytokine signalling.

Whether two sequences are homologous is routinely calculated using a percentage similarity or identity, terms that are well known in the art. Homologues preferably have 70% or greater similarity or identity at the nucleic acid or amino acid level, more preferably 80% or greater, more preferably 90% or greater, such as 95% or 99% identity or similarity at the nucleic acid or amino acid level. A number of programs are available to calculate similarity or identity; preferred programs are the BLASTn, BLASTp and BLASTx programs, run with default parameters, available at www.ncbi.nlm.nih.gov. For example, two nucleotide sequences may be compared using the BLASTn program with default parameters (score=100, word length=11, expectation value=11, low complexity filtering=on). The above levels of homology are calculated using these default parameters.

The skilled person will realise that a gene or gene product identified in a patient may differ slightly from the exact gene or product sequence provided herein, yet is still recognisable as the same gene or gene product. Any gene or gene product that is recognisable by a skilled person as the same as one referred to herein, is within the scope of the invention. For example, a skilled person may identify a polynucleotide or polypeptide under investigation by a partial sequence and/or a physical characteristic, such as the molecular weight of the gene product. The gene or gene product in a patient may be an isoform of that defined herein. Accordingly, isoforms and splice variants are within the scope of the present invention. The skilled person will realise that differences in sequences between individuals, for example single nucleotide polymorphisms, are within the scope of the invention. The key to the invention is that the polynucleotide or polypeptide that is identified in a sample isolated from a patient is recognisable as one characterised herein.

TABLE 1

| SEQ | Common | GenBank | |
|---|---|---|---|
| ID No. | Name | Accession No. | Description |
| 1 | ELN | NM_000501 | Elastin (supravalvular aortic |
| | | | stenosis, Williams-Beuren |
| | | | syndrome) |
| 2 | RGS-1 | NM_002922 | Regulator of G-protein signal- |
| | | | ling 1 |
| 3 | SOCS-3 | NM_003955 | Suppressor of cytokine signal- |
| | | | ling 3 |
| 4 | PTGS-2 | NM_000963 | Prostaglandin-endoperoxide |
| | | | synthase 2 (prostaglandin G/H |
| | | | synthase and cyclooxygenase) |
| 5 | JUN | NM_00228.3 | Oncogene Jun |
| 6 | ATF-3 | NM_001040619.1 | Activating transcription fac- |
| | | | tor 3 |
| 7 | CTGF | NM_001901 | Connective tissue growth fac- |
| | | | tor |
| 8 | IGF-2 | NM_000612.4 | Insulin-like growth factor 2 |
| | | | (Somatomedin A) |
| 9 | RBMS-1 | NM_016836 | RNA binding motif, single |
| | | | stranded interaction protein 1 |
| | | | (Transcript variant 1) |
| 10 | RBMS-1 | NM_016839 | RNA binding motif, single |
| | | (Previously known | stranded interaction protein 1 |
| | | as NM_016837) | (Transcript variant 2) |
| 11 | RBMS-1 | NM_002897 | RNA binding motif, single |
| | | | stranded interaction protein 1 |
| | | | (Transcript variant 3) |

The marker genes of the invention were identified by comparing gene expression patterns between colorectal tissue obtained from normal, non-cancer patients (normal normal) and the “normal” (i.e. non-cancerous) tissue adjacent to cancerous colorectal tissue (adjacent normal).

A IIlumina micro-array technology of >35,000 genes was used to obtain separate RNA expression profiles for normal normal (NN) and adjacent normal (AN) tissue biopsies. Appropriate software was used to identify differentially expressed genes between the two tissue types. This revealed an extensive list of genes that were both up-regulated and down-regulated in the NN samples. The results were presented as a duff score and a p-value, which give an indication of the degree of up-regulation or down-regulation of each gene in the NN samples. A p-value <0.05 was considered significant.

Each of the genes identified was researched for possible implications in colorectal cancer. A total of 62 genes which, according to the micro-array data, showed the highest levels of differential expression between the NN and AN samples (the highest positive and negative diff score) and which revealed genes with known biological roles in cancer or in relevant interconnecting pathways were selected. Each of these differentially expressed genes was then validated and their precise expression levels determined in a progressively larger cohort of samples from the two groups using fully quantitative RT-qPCR to establish the colorectal cancer risk index based on a specific, validated, gene signature obtained from the differentially expressed RNAs.

Therefore, this methodology identified genes that are up-regulated in the non-cancerous tissue of cancer patients, compared with the corresponding tissue from healthy individuals. The increased expression of these genes therefore indicates a predisposition to cancer, in particular colorectal cancer.

As used herein, the term “gene product” refers to the mRNA or polypeptide product that results from transcription and/or translation of the gene. The methods to carry out the diagnosis can involve the synthesis of cDNA from the mRNA in a test sample, amplifying as appropriate, portions of the cDNA corresponding to the genes or fragments thereof and detecting each product as an indication of the risk of the disease in that tissue, or detecting translation products of the mRNAs comprising gene sequences as an indication of the risk of the disease.

Preferably, the actual level of expression (mRNA copy number) of all the nine genes is divided by the expression levels of two constitutively expressed reference genes, which are expressed at the same level in each tissue and have no known function in any disease. This minimises inter-assay variations. Examples of suitable genes include S100A16 (Homo sapien S100 calcium being protein A16; GenBank Accession No. NM_{—}080388) and CEBP (Homo sapien CCAAT/enhancer binding protein CC/EBP) alpha; NM_{—}004364.3).

FIG. 1 shows that the relative expression of each of the nine genes of the invention, normalised using reference genes, is up-regulated in the adjacent normal (AN) tissue, compared to the normal normal (NN) tissue.

As shown in FIG. 2, the Cancer Risk Index (CRI) calculated using the combined expression of the nine genes in AN tissue is higher than the CRI for the NN tissue sample (a p-value <0.001 was considered significant). Therefore the result illustrated in this graph is indicative of colorectal cancer in the patients from whom the AN samples were taken.

The level of expression of each of the nine genes or gene products in the patient can be detected in vivo or ex vivo. In a preferred embodiment, expression is detected ex vivo, in a sample of genetic material that is isolated from the patient. The sample material is preferably isolated from colorectal tissue. As the combination of nine genes or their gene products is useful as a marker for the risk of cancer, it is preferred that the tissue sample is not already cancerous. Therefore, a preferred tissue is non-cancerous colorectal tissue. The tissue may be obtained by any suitable means, for example by biopsy. Alternatively, expression of the marker genes can be determined in vivo, for example using techniques such as “Quantum Dot” labelling. If the method is carried out in vivo, gene expression is preferably determined in colorectal tissue.

Highly luminescent “Quantum Dots”, which are known in the art, are highly stable against photo-bleaching and have narrow, symmetric emission spectra.

The emission wavelength of quantum dots can be continuously tuned by changing the particle size or composition, and a single light source can be used for simultaneous excitation of all different-coloured dots. Bio-conjugated quantum dots typically comprise a collection of different sized nanoparticles embedded in tiny beads of polymer material. These can be finely tuned to various luminescent colours that can be used to label one or more sequences that hybridise to genes identified herein as predictive for cancer risk. The quantum dot labelled sequences can be targeted to the colon or rectum using techniques known to the skilled person, for example using an antibody that is specific to a protein that is expressed in the colorectal tissue. For example, a conjugated anti-guanylyl cyclase C receptor antibody will target the quantum dot-labelled sequences to the colon following injection into the bloodstream. A number of other techniques for delivering quantum dot labelled marker sequences to colorectal cells will be apparent to the skilled person, including the use of translocation peptides, liposomes and endocytic uptake. One preferred system is based on the use of small cyclic repeating molecules of glucose known as cyclodextrins, which are assembled into linear cyclodextrin-containing polymers. These can be synthesised over a broad range of molecular weights, providing tuneable properties for marker delivery that improve localisation at the target tissue. Another preferred approach coats quantum dots with a polymer such as polyethylene glycol) (PEG), and attaches these coated dots to a homing peptide (e.g. guanylyl cyclase c receptor) and one or more specific markers targeting the genes identified in Table 1, thereby forming a nanoparticle. As binding to the target (colorectal) tissue occurs, the nanoparticle is taken up by the colonic cells and the oligonucleotide probes bind to their target complementary RNA. Since each marker is associated with a specific quantum dot emitting fluorescence at a specific wavelength, both intensity and spectrum of emission are indicative of successful hybridisation and presence of target mRNA.

If the individual's colon expresses the specific gene(s) to which a marker-quantum dot conjugate is complementary, the quantum dots will hybridise to their targets within the colon and emit light at a characteristic wavelength. This will result in a colour signal for real-time “optical biopsy”. The quantum dots can be detected by infra-red optical imaging in vivo, for example in the colon, directly through the tissue or by using a colonoscope allowing a real-time optical “biopsy”. This procedure would result in a diagnosis without tissue removal. This technique can also be used to monitor a diagnosis or treatment.

The present invention is also directed to the use of a combination of nine isolated genes identified herein as ELN, RGS-1, SOCS-3, PTGS-2, JUN, ATF-3, CTGF, IGF-2 and RBMS-1 in an ex vivo diagnostic assay to test for the risk of cancer, preferably colorectal cancer, in a patient.

A further embodiment of the invention provides a kit for the detection of the risk of cancer in a patient, comprising a combination of reagents that bind to each of the genes identified herein as ELN, RGS-1, SOCS-3, PTGS-2, JUN, ATF-3, CTGF, IGF-2 and RBMS-1 and instructions for detecting the risk of cancer.

Useful reagents for inclusion in said kit include polynucleotides comprising the isolated gene sequences identified herein as SEQ ID Nos. 1-8 and at least one of SEQ ID Nos. 9-11, their complements, or fragment(s) thereof which may be useful in diagnostic methods such as RT-PCR, PCR or hybridisation assays of mRNA extracted from biopsied tissue, blood or other test samples; or proteins which are the translation products of such mRNAs; or antibodies directed against these proteins.

Identification of the nine genes of the invention, or their expressed products, may be carried out by techniques known for the detection or characterisation of polynucleotides or polypeptides. For example, isolated genetic material from a patient can be probed using short oligonucleotides that hybridise specifically to the target gene. The oligonucleotide probes may be detectably labelled, for example with a fluorophore, so that upon hybridisation with the target gene, the probes can be detected. Alternatively, the gene, or parts thereof, may be amplified using the polymerase enzyme, e.g. in the polymerase chain reaction, with the amplified products being identified, again using labelled oligonucleotides.

Diagnostic assays incorporating any of the genes, proteins or antibodies according to the invention will include, but are not limited to:
Polymerase chain reaction (PCR)
Reverse transcription PCR
Real-time PCR
In-situ hybridisation
Southern dot blots
Immuno-histochemistry
Ribonuclease protection assay
cDNA array techniques
ELISA
Protein, antigen or antibody arrays on solid supports such as glass or ceramics
Small interfering RNA functional assays.

All of the above techniques are well known to those in the art. Preferably, the diagnostic assay is carried out ex vivo, outside of the body of the patient.

The preferred diagnostic technique is Real-time PCR. Real-time PCR, also known as kinetic PCR, qPCR, qRT-PCR and RT-qPCR, is a quantitative PCR method for the determination of copy numbers of templates such as DNA or RNA in a PCR reaction. There are two kinds of Real-time PCR: probe-based and intercalator-based. Both methods require a special thermocycler equipped with a sensitive camera that monitors the fluorescence in each reaction at frequent intervals during the PCR reaction. Probe-based real-time PCR, also known as TaqMan PCR, requires a pair of PCR primers (as in regular PCR) and an additional fluorogenic probe which is an oligonucleotide with both a reporter fluorescent dye and a quencher dye attached. The intercalator-based method, also known as the SYBR Green method, requires a double-stranded DNA dye in the PCR reaction which binds to newly synthesised double-stranded DNA and gives fluorescence.

The identification of the genes in Table 1 also permits therapies to be developed, with each gene being a target for therapeutic molecules. For example, there are now many known molecules that have been developed for gene therapy, to target and prevent the expression of a specific gene. Molecules of particular interest are small interfering RNA (siRNA) molecules and micro RNA (miRNA) molecules. Small interfering RNA (siRNA) suppresses the expression of a specific target protein by stimulating the degradation of the target mRNA. Micro RNA's (miRNA's) are single stranded RNA molecules of about 20 to 25, usually 21 to 23, nucleotides that are thought to regulate gene expression. Other synthetic oligonucleotides are also known which can bind to a gene of interest (or its regulatory elements) to modify expression. Peptide nucleic acids (PNAs) in association with DNA (PNA-DNA chimeras) have also been shown to exhibit strong decoy activity, to alter the expression of the gene of interest. Molecules, preferably polynucleotides, that can alter the expression level of a gene identified in Table 1 are therefore useful in the prevention and treatment of cancer, preferably colorectal cancer, and are within the scope of the invention. The skilled person will realise whether up-regulation or down-regulation (inhibition) of each gene is required.

The present invention also includes antibodies raised against a peptide of any of the genes identified in the invention. The term “antibody” refers broadly to any immunologic binding agent such as IgG, IgM, IgA, IgD and IgE. An antibody binds, preferably specifically, to an antigen. Antibody is also used to refer to any antibody-like molecule that has an antigen-binding region and includes antibody fragments such as single domain antibodies (DABS), Fv, scFv, aptamers, etc. The techniques for preparing and using various antibody-based constructs and fragments are well known in the art. Means for preparing and characterising antibodies are also well known in the art.

The antibodies will usually have an affinity for the peptide, encoded by a gene identified in Table 1, of at least 10⁻⁶M, more preferably, 10⁻⁹M and most preferably at least 10⁻¹¹M. The antibody is preferably specific to the peptide of the invention, i.e. it binds with high affinity only to a specific peptide of the invention, and does not bind to other peptides. This allows the antibody to bind specifically to the peptide of the invention in a mixture containing a number of different peptides. The antibody may be of any suitable type, including monoclonal or polyclonal. Combinations of antibodies to each of the peptides encoded by genes according to Table 1 are within the scope of the invention.

Assay kits for determining the presence of each peptide antigen in a test sample are also included. In one embodiment, the assay kit comprises a container comprising antibodies that specifically bind to the antigens, wherein the antigens comprise at least one epitope encoded by each gene identified in Table 1. As such, the kit contains antibodies to epitopes encoded by multiple genes according to Table 1 and the different antibodies can be packaged together (in a single container), or separately, within the kit. These kits can further comprise containers with useful tools for collecting test samples, such as blood, saliva, urine and stool. Such tools include lancets and absorbent paper or cloth for collecting and stabilising blood, swabs for collecting and stabilising saliva, cups for collecting and stabilising urine and stool samples. The antibody can be attached to a solid phase, such as glass or a ceramic surface.

Detection of antibodies that bind specifically to each of the antigens in a test sample suspected of containing these antibodies may also be carried out. This detection method comprises contacting the test sample with polypeptides, containing at least one epitope of each gene identified in Table 1. Contact is performed for a time and under conditions sufficient to allow antigen/antibody complexes to form. The method further entails detecting complexes, which contain the polypeptides encoded by SEQ ID Nos. 1-8 and at least one of SEQ ID Nos. 9-11. The polypeptide complex can be produced recombinantly or synthetically or be purified from natural sources.

If desired, the cancer screening methods of the present invention may be readily combined with other methods in order to provide an even more reliable indication of diagnosis or prognosis, thus providing a multi-marker test.

1. An ex vivo method for detecting the risk of cancer in a patient, comprising the step of:
i) detecting the expression level of the genes identified herein as ELN, RGS-1, SOCS-3, PTGS-2, JUN, ATF-3, CTGF, IGF-2 and RBMS-1, in a sample of genetic material isolated from a patient,
wherein the combined expression level indicates the risk of cancer in the patient from whom the sample was isolated.

2. A method according to claim 1, wherein the expression level of each gene is combined to produce a combined expression value.

3. A method according to claim 2, wherein the combined expression value is compared with a control value in order to determine whether the patient is at risk of cancer.

4. A method according to claim 3, wherein a combined expression value higher than the control value indicates that the patient is at risk of cancer.

5. A method according to claim 3, wherein the control value is a pre-determined value.

6. A method according to claim 1, wherein the expression level of each gene is compared to the expression level of the corresponding gene from a control sample.

7. A method according to claim 6, wherein an increase in the expression level of each of the genes, compared to the corresponding control, indicates a risk of cancer in the patient from whom the sample was isolated.

8. A method according to claim 6, wherein the control sample is genetic material isolated from a healthy individual.

9. A method according to claim 1, wherein the genes to be detected in the patient's sample are identified as SEQ ID Nos. 1-8 and at least one of SEQ ID Nos. 9-11, or the complement thereof, or polynucleotides of at least 10 consecutive nucleotides that hybridise to the sequences (or the complement thereof) under stringent hybridising conditions.

10. A method according to claim 1, wherein the sample of genetic material isolated from the patient is non-cancerous colorectal tissue.

11. A method according to claim 1, wherein the cancer is colorectal cancer.

12. Use of a combination of nine isolated genes identified herein as ELN, RGS-1, SOCS-3, PTGS-2, JUN, ATF-3, CTGF, IGF-2 and RBMS-1 in an ex vivo diagnostic assay to test for the risk of cancer in a patient.

13. Use according to claim 12, wherein the isolated genes are identified herein as SEQ ID Nos.1-8 and at least one of SEQ ID Nos. 9-11, or the complement thereof, or polynucleotides of at least 10 consecutive nucleotides that hybridise to the sequences (or the complement thereof) under stringent hybridising conditions.

14. Use according to claim 12, wherein the cancer is colorectal cancer.

15. A kit for the detection of the risk of cancer in a patient, comprising a combination of reagents that bind to each of the genes identified herein as ELN, RGS-1, SOCS-3, PTGS-2, JUN, ATF-3, CTGF, IGF-2 and RBMS-, and instructions for detecting the risk of cancer.

16. A kit according to claim 15, wherein the reagents bind to genes identified herein as SEQ ID Nos. 1-8 and at least one of SEQ ID Nos. 9-11, or bind to the complement thereof, or polynucleotides of at least 10 consecutive nucleotides that hybridise to the sequences (or a complement thereof) under stringent hybridising conditions, or peptides encoded by said genes, gene complements or fragments.

17. A kit according to claim 15, wherein the reagents are antibodies that bind to peptides encoded by said genes.

18. A kit according to claim 15, wherein the reagents are polynucleotides that hybridise to said genes.

19. A kit according to any of claim 15, further comprising quantum dots.

20. An in vivo method for the detection of the risk of cancer in a patient, comprising the step of:
(i) detecting the expression level of the genes identified herein as ELN, RGS-1, SOCS-3, PTGS-2, JUN, ATF-3, CTGF, IGF-2 and RBMS-1 in a patient,
wherein the expression level indicates the risk of cancer in the patient.

21. A method according to claim 20, wherein the expression level of each gene is combined to produce a combined expression value.

22. A method according to claim 21, wherein the combined expression value is compared with a control value in order to determine whether the patient is at risk of cancer.

23. A method according to claim 22, wherein a combined expression value higher than the control value indicates that the patient is at risk of cancer.

24. A method according to claim 22, wherein the control value is a pre-determined value.

25. A method according to claim 20, wherein the expression level of each gene is compared to the expression level of the corresponding gene from a control sample.

26. A method according to claim 25, wherein an increase in the expression level of each of the genes, compared to the corresponding control, indicates a risk of cancer in the patient.

27. A method according to claim 25, wherein the control sample is genetic material isolated from a healthy individual.

28. A method according to any of claim 20, wherein the genes to be detected in the patient are identified as SEQ ID Nos. 1-8 and at least one of SEQ ID Nos. 9-11, or the complement thereof, or polynucleotides of at least 10 consecutive nucleotides that hybridise to the sequences (or the complement thereof) under stringent hybridising conditions.

29. A method according to any of claim 20, wherein the sample of genetic material isolated from the patient is non-cancerous colorectal tissue.

30. A method according to any of claim 20 wherein the cancer is colorectal cancer.
